(19) United States
(12) Patent Application Publication
(10) Pub. No.: US 2004/0009211 A1
(43) Pub. Date: Jan. 15, 2004

(54) Film-dermatics

(76) Inventors: Michael Roreger, Neuwied (DE); Iris Schnitzler, Bonn (DE); Armin Wadle, Erkrath (DE); Bernhard Bandowski, Dusseldorf (DE)

Correspondence Address: BIRCH STEWART KOLASCH & BIRCH, PO BOX 747, FALLS CHURCH, VA, 22040-0747, US

(21) Appl. No.: 10/333,027

(22) PCT Filed: Jun. 30, 2001
(86) PCT No.: PCT/EP01/07504

(30) Foreign Application Priority Data
Jul. 15, 2000 (DE) 10034491.7

(51) Int. Cl. A61K 9/70; A61K 7/00

(52) U.S. Cl. 424/449; 424/401

(57) ABSTRACT

The invention relates to an administration form for delivering pharmaceutical and/or cosmetic active substances to the skin in homogeneous distribution. The inventive administration form is a flexible active substance-containing film which upon contact with water forms a spreadable solution, dispersion or emulsion on the skin and releases the active substance(s) in a defined dose.

The invention relates to sheet-like or film-like administration forms for delivering pharmaceutical or cosmetic active substances to the skin, preferably to the human skin. Active agents for skin care, for treatment of skin diseases or subcutaneous diseases, such as rheumatism, are usually administered with the aid of solutions, lotions, powders, sprays or semi-solid preparations, such as ointments, creams or gels. Such administration forms are typically provided in multiple-dose containers, such as tubes or crucibles.

This means that in such a container there is contained a quantity of the administration form which is intended for a plurality of applications involving appropriate dosage processes. The dosing itself is performed individually by the user. The user will only be able to assess the amount of active agent dosed if he weighs the respective dose prior to application. If the application is repeated, the reproducible application of a constant amount of active substance would be possible only by way of a preceding weighing process. This individually variable dosage process is possible only because of the low coherence and ready separability of these administration forms. On the other hand, their low coherence offers the advantage that the administration form, as mentioned, can be deformed to any shape whatever and conformed to uneven surfaces.

During the life of a product, the container will frequently be opened and closed again. Since there will automatically occur contamination of the administration form by air-borne organisms, such administration forms need to be preserved and protected from microbial decay.

Disadvantages of such administration forms are, therefore, that users suffering from allergy to preservatives are not able to use a large number of products, and that accurate and reproducible dosing of highly efficacious substances, for instance, is not possible.

The disadvantages mentioned hereinbefore can be avoided by employing dry administration forms in the form of films or sheets, sponges, cloths or nonwoven sheets which are moistened and activated prior or subsequent to application thereof to the skin. Such administration forms are described, for example, in JP 110 49 635, JP 580 21 608 or JP 8188 527.

Such administration forms can be formulated free of preservatives, and they enable accurate and reproducible dosing of active substances. The disadvantages of these administration forms are that they are not soluble and therefore not spreadable, that the surface to be treated is predetermined by the dimensions of the administration form and that it is not possible to apply, for example, complex emulsion systems by means of such administration forms. The object of the present invention is therefore to find an individually packable administration form for delivery of a defined single dose of active agents to the skin which avoids the disadvantages of administration forms according to the prior art.

Surprisingly, the solution was found to lie in a flexible active substance-containing film or sheet which when placed on the skin forms a spreadable solution, dispersion or emulsion upon contact with skin moisture and/or perspiration and immediately releases a single dose of the active substance or of a plurality of active substances. The administration form can be formulated to be free of preservatives since it is present as a dried film, and it has the prop rty that it is possible to apply and distribute a defined quantity of active agent over a desired area of application as many times as desired and in a reproducible manner.

In a preferred embodiment, the film is made up of

1-60% of at least one film former soluble in polar solvents, preferably in water or polar oganic solvents such as ethanol, isopropanol or ethyl acetate, or mixtures thereof,

1-60% of at least one water-soluble gelling agent

1-60%-wt. of at least one plasticizer, and

0.1-40%-wt. of at least one active substance,

and optionally further auxiliary substances.

In a particularly preferred embodiment, the film is comprised of

5-50%-wt. of at least one film former soluble in polar solvents,

1-50%-wt. of at least one gelling agent,

0.5-50%-wt. of at least one plasticizer,

0.5-40%-wt. of at least one active substance,

and optionally further auxiliary substances.

The type and quantity of the film former determine the strength and stability of the film in its dried state. To produce the film, initially, a flowable solution, dispersion or emulsion must be formulated, from which, by way of spreading and drying, the solvent or dispersing medium is removed. To make this process as short as possible, easily withdrawable solvents such as water, ethanol, isopropanol, ethyl acetate or mixtures thereof are preferred for making the base mass.

Suitable polymeric film formers soluble in the organic polar solvents are preferably found among the polyvinyl pyrrolidones, the polyvinyl alcohols, the polyacrylic acids and polymethacrylic acids, the celluloses, the derivatives thereof, as well as their combinations.

To enable that upon application to the skin a ready-to-use preparation in the form of, for example, a solution or lotion, a gel or a cream may be formed from the dried film by way of contact with water or skin perspiration, the film contains one or more water-soluble gelling agents, which ensure that the film will swell and disintegrate spontaneously when it comes into contact with moisture. The consistency of the spontaneously forming administration form, which immediately after its formation releases active agent to the skin, is directly dependent on the type and concentration of the gelling agents employed, and on the amount of water or aqueous solution, such as perspiration, available. Thus, for example, both an aqueous solution and a hydrogel can be formed from a given film formulation. Suitable water-soluble gelling agents are preferably natural or semi-synthetic polymers from the group of plant polysaccharides such as, for instance, alginates, pectins, carrageenans, tragacanth or xanthene, cellulose derivatives such as methyl cellulose, hydroxpropyl cellulose, hydroxethyl cellulose, hydroxypropyl methyl cellulose or sodium carboxymethyl cellulose, starch and starch derivatives, galactoglucomannan and galactoglucomannan derivatives, chitosan and chitosan derivatives, as well as combinations thereof.

On principle, the film-shaped administration forms according to the present invention must not be rigid or brittle since otherwise it would be considerably more difficult to use the film on an uneven surface such as the skin. To guarantee sufficient flexibility, the film contains plasticizers such as, for example, glycerol, sorbitol, mannitol, low-molecular polyethylene glycols and polypropylene glycols, citric acid esters such as triethyl citrate or acetyltriethyl citrate, tartaric acid esters such as dibutyl tartrate, glycerol esters such as glyceryl diacetate or glyceryl triacetate, phthalic acid esters such as dibutyl phthalate or diethyl phthalate and/or hydrophilic tensides, preferably hydrophilic, non-ionogenic tensides such as, for instance, partial fatty acid esters of sugars, polyethylene glycol fatty acid esters, polyethylene glycol fatty alcohol ethers or polyethylene glycol-sorbitan fatty acid ethers or polyethylene glycol-sorbitan fatty acid esters.

The inventive administration form for releasing active agents to the skin may contain pharmaceutically active agents for dermal treatment of local skin diseases or for intradermal treatment of diseases, as well as cosmetic active agents for skin care or for exerting an influence on skin conditions. For dermal treatment of local skin diseases, local anaesthetics, local antibiotics, antiseptics, antimycotics, antihistaminics, and antipruritic drugs, keratolytics and caustic drugs, virustatics, antiscabetic agents, steroids, as well as various substances for treating acne, psoriasis or photodermatoses are used. The active substances which according to the invention are applied intradermally comprise: steroid and non-steroid antirheumatics, local anaesthetics, substances stimulating the blood flow, vasoprotectors or vasoconstrictors for treatment of vascular diseases as well as active agents for influencing processes in the subcutaneous fatty tissue.

For cosmetic applications the administration form according to the present invention may contain, for example, active agents for treating wrinkles, aged skin, impure skin rashes, for lightening the skin, for moisturizing the skin, pimples, for regeneration and revitalisation, for skin tightening, for light protection, for reducing perspiration, for n utralizing and covering odours, for depilation, for cleansing and personal hygiene, as well as volatile active substances for protection against mosquitoes, wasps or ticks.

In a particular embodiment, initially, an oil-in-water emulsion is made in a conventional manner known to those skilled in the art; from this emulsion is formed, by way of spreading and drying, a film containing an internal phase present in small droplets. When the film has dried, this internal lipophile phase is immobilised by the polymeric network of the hydrophilic external phase such that there is a very low tendency for phase separation during the storage of the film. In addition, the administration form according to the present invention offers the advantage that the use of emulsion-stabilizing auxiliary substances can be markedly reduced.

The lipophile phase may consist of natural, semi-synthetic or synthetic fats and oils such as olive oil, caster oil, peanut oil, soy oil, linseed oil, sesame oil, jojoba oil, avocado oil, hydrogenated peanut oil, hydrogenated caster oil, triglyceride mixtures (Miglyolo, Softisan®), or silicone oils, natural, semi-synthetic or synthetic waxes such as bees wax, wool wax, earth wax, spermaceti, oleic acid oleyl ester, isopropyl palmitate, isopropyl myristate, ethyl oleate, cetyl palmitate or cetyl stearate, fatty alcohols such as dodecyl alcohol or cetyl alcohol, fatty acids such myristic acid, oleic acid or linoleic acid, propoxylated, ethoxylated or sulfated fatty alcohols, fatty acid alkyl amides, fatty acid-protein condensation products, phospholipids, sterols, or carbohydrates such as paraffins or paraffin oils.

In addition to the auxiliary substances already m ntioned, the administration form for delivery of active substances may contain the following as auxiliary substances:

penetration enhancers such as alkyl sulfates, alkyl sulfonates, alkali soaps, fatty acid salts of polyvalent metals, betaines, amine oxides, fatty acids, fatty acid esters, mono-, di- or triglycerides, long-chain alcohols, sulfoxides, nicotinic acid esters, salicylic acid, N-methyl pyrrolidone, 2-pyrrolidone, or urea

preserving agents such as, for example, p-C1-m-cresol, phenyl ethyl alcohol, phenoxyethyl alcohol, chlorobutanol, 4-hydroxybenzoic acid methyl ester, 4-hydroxybenzoic acid propyl ester, benzalkonium chloride, cetyl pyridinium chloride, chlorohexidine diacetate or gluconate, ethanol or propylene glycol,

pH regulators such as, for example, glycerol buffers, citrate buffers, borate buffers, phosphate buffers or citric acid-phosphate buffers

antioxidants such as, for example, ascorbic acid, ascorbyl palmitate, tocopherol acetate, propyl gallate, butyl hydroxyanisole or butyl hydroxytoluene

emulsion stabilizers such as, for example, nonionogenic emulsifiers, amphoteric emulsifiers, cationactive emulsifiers, and anion-active emulsifiers

fillers such as, for example, micro-crystalline cellulose, sodium oxide, zinc oxide, titanium oxide, talcum, silicon dioxide, magnesium silicate, magnesium aluminium silicate, kaolin, hydrophobic starch, calcium stearate or calcium phosphate.

To produce an inventive administration form, initially a low-viscous flowable mass, e.g. a solution, a dispersion or an emulsion which contains active agent in homogeneously distributed form is prepared. This mass is then used to coat a sheet-like substrate, which has been rendered abhesive, by employing a method known to the person skilled in the art. Solidification takes place after coating of the sheet-like substrate by withdrawing solvent or dispersing medium by means of drying. The manner and extent to which cohesive forces are built up during this process is dependent on the polymer backbone of the film formed by the film former and the gelatinizing agent. The result is a broad, film-shaped continuous tape or web having a thickness predetermined by the coating. A limiting factor for the thickness of the web in a given formulation is the need for flexibility and deformability of the individual, separated administration forms, so as to enable them to be adapted to the surface of the skin. Separation of the individual administration forms having a predetermined surface area from the continuous web is performed according to known methods, such as punching and cutting. After separating the films they can be packed individually in small bags or severally in appropriate film dispenser systems. Since the coating with the mass, which contains active agent in homogeneously distributed form, is performed maintaining a constant coating weight, all the separated individual administration forms contain the same amount of active substance in homogeneous distribution. This enables the user to dose in an accurate and reproducible manner.

Since the active substance content per unit area and surface is stepless variable within broad limits by the production process, the administration form according to the present invention offers the possibility of dosing even very small quantities of active substances in an accurate and reproducible manner.

According to a preferred embodiment of the invention it is provided that the film is produced from a solution, dispersion and/or emulsion by dosing into sheets or films provided with small cups (e.g. thermoformed blisters) and subsequent drying. The invention thus comprises administration forms of the aforementioned kind, wherein the active substance-containing sheet or film can be obtained from a solution, dispersion and/or emulsion by dosing into films provided with cups (e.g. thermoformed blisters) and subsequent drying.

For the user, the packing of the inventive administration form in small bags or film dispensers offers the advantage that such packaging units can be accommodated in clothing and purses in a simple and space-saving manner. By contrast to administration forms in multiple-dose containers such as tubes and crucibles, in the case of the individually dosed film, neither microbial contamination nor loss of active compound, caused by frequent opening and closing, can occur.

Possible formulations and processes for preparing the inventive film for delivery of active agent to the skin according to the features of the main claims will be explained in the following by way of example, without thereby limiting the invention.

### EXAMPLE 1

Stirring evenly, 4 g polyvinyl alcohol (Mowiol 8-88) and 6 g hydroxypropyl cellulose (Klucel LF) are dissolved in a mixture of 50 g isopropanol, 15 g ethyl acetate and 10 g water. Then, 2 g carrageen, 3 g calcium-modified corn starch (Dry Flo AF), 6 g glycerol, 3.6 g polyethylene glycol 400 and 0.4 g lidocaine hydrochloride are stirred in, until a homogeneous dispersion is reached.

The dispersion is coated with a coating thickness of 400 μm on a siliconised paper, and is dried conv ctively in a drying channel at 60° C. and air velocity of approx. 5 m/sec. After drying, a soft, deformable film is obtained, which has a weight per unit area of 100 g/m². From the dried web, rectangular film sections of an area of 30 cm² are cut. After application on the skin and moistening with water, a hydrogel forms spontaneously, which, after rubbing in at the application site, develops local anaesthetic action required, for example, in the treatment of pain caused by tennis elbow.

### EXAMPLE 2

Stirring evenly, 8 g polyvinyl pyrrolidone (Kollidon 90) are dissolved in a mixture of 44 g ethanol, 16 g ethyl acetate and 10 g water. Then, 6 g sodium carboxymethyl cellulose (Walocel 10000), 8 g glycerol, 2 g sorbitol, 3 g echinacea tincture and 3 parts chamomile extract are stirred in until a homogeneous distribution is obtained.

The solution is spread at a coating thickness of 400 μm onto a siliconized paper, and convectively dried in a drying channel at 60° C. and air velocity of approx. 5 m/sec. After drying, a soft, deformable film is obtained, which has a weight per unit area of 120 g/m².

From the dried web, oval film sections of 16 cm² are punched out. After application to the skin and moistening with water, a hydrogel forms spontaneously which is used for local treatment of sunburn and other first-degree burns, contused injuries and slow-healing superficial wounds.

### EXAMPLE 3

Stirring evenly, 2 g polyacrylic acid (Carbopol 940) and 4 g ethyl cellulose (Ethyl Cellulose N 50 NF) are dissolved in a mixture of 60 g ethanol and 10 g water. Then, 2 g sodium alginate (Manucol LB), 3 g tapioca (Tapioca pure 28-180.), 5 g glycerol, 5 g polyethylene glycol 400, 4 g arnica tincture and 5 g calendula tincture are stirred in, until a homogenous dispersion is obtained.

This dispersion is coated at a coating thickness of 350 μm onto a siliconized paper, and convectively dried in a drying channel at 60° C. and an air velocity of approx. 5 m/sec. After drying, a soft, deformable film is obtained, which has a weight per unit area of 105 g/m².

From the dried web, rectangular film sections of 25 cm² area are punched out. After application to the skin and moistening with water, a hydrogel forms spontaneously which is used for intradermal treatment of sprains, contusions and haemorrhages.

### EXAMPLE 4

Stirring evenly, 6 g polyvinyl pyrrolidone (Kollidon 90) and 2 g ethyl cellulose (Ethyl Cellulose N 50 NF) are dissolved in a mixture of 55 g ethanol and 10 g ethyl acetate. Subsequently, 2 g sodium alginate (Manucol LB), 6 g sodium carboxymethyl cellulose (Walocel 10000), 4 g glycerol, 6 g sorbitol, 0.5 g salicylic acid, 0.5 g chlorohexidinedigluconate solution 20%, 3 g salvia extract and 5 g chamomile tincture are stirred in, until a homogeneous dispersion is obtained.

This dispersion is spread at a coating thickness of 360 μm onto siliconised paper, convectively dried in a drying channel at 60° C. and an air velocity of approx. 5 m/sec. After drying, a soft, deformable film is obtained, which has a weight per unit area of 120 g/m².

From the dried web, circular film sections of an area of 10 cm² are punched out. After application to the skin and moistening with water, a hydrogel forms which has desiccant, keratolytic and anti-inflammatory action and can therefore be employed in the treatment of acne.

### EXAMPLE 5

10 g hydroxpropyl methyl cellulose, 9 g talcum, 5 g sodium starch-octenyl succinate (Fry Flo Plus), 10 g glycerol, 8 g sodium chlorohydrate (Chlorhydrol), 0.2 g phenoxyethanol, 1 g dimethicon fluid, 0.5 g Pluronic F68 and 0.2 g perfume oil are stirred into 64.1 g water, until a homogeneous dispersion is obtained.

This dispersion is spread at a coating thickness of 250 μm onto a siliconized paper, and convectively dried in a drying channel at 70° C. and an air velocity of approx. 8 m/sec. After drying, a soft, deformable film is obtained, which has a weight per unit area of 80 g/m².

From the dried web, rectangular film sections of 10 cm² area are punched out. When perspiration occurs, film sections are applied, for instance, in the armpits. They dissolve spontaneously and have an emphractic and odourneutralising effect

### EXAMPLE 6

6 g hydroxypropyl cellulose (Klucel LF), 10 g glycerol and 12 g calcium-modified corn starch (Dry Flo AF) are dissolved in 55.9 g water by stirring evenly and heating to 65° C. (Phase A).

8 g soy oil, 2 g polyethylene glycol monostearate, 4 g cetyl stearyl alcohol, 2 g tocopherol acetate and 0.1 g retinyl palmitate are mixed and slowly stirred, while heating, until a clear solution is obtained (Phase B). Phase A is incorporated in portions in Phase B at 65° C. by stirring and homogenizing.

This emulsion is coated at a temperature of 65° C. and at a coating thickness of 250 μm onto a siliconized paper, and convectively dried in a drying channel at 70° C. and an air velocity of approx. 8 m/sec. After drying, a soft, deformable film is obtained, which has a weight per unit area of 120 g/m².

From the dried web, oval film sections of 20 cm² are punched out. After application to the skin and moistening with water, an oil-in-water emulsion forms, which can be used, for example, as a night cream for regeneration and revitalisation of the skin by delivery of vitamins.

1. Administration form for delivering pharmaceutical and/or cosmetic active substances to the skin in homogeneous distribution, characterized in that the said administration form is a flexible active substance-containing film or sheet which upon contact with water forms a spreadable solution, dispersion or emulsion on the skin and contains the active substance(s) in a defined dose.

2. Administration form according to claim 1, characterized in that the said film or sheet is made up of
1-60%-wt. of at least one film former soluble in polar solvents
1-60%-wt. of at least one water-soluble gelling agent,
1-60%-wt. of at least one plasticizer, and
0.1-40%-wt. of at least one active substance,
and optionally further auxiliary substances.

3. Administration form according to claim 2, characterized in that the said film or sheet is made up of
5-50%-wt. of at least one film former soluble in polar solvents,
1-50%-wt. of at least one gelling agent,
0.5-50%-wt. of at least one plasticizer, and
0.5-40%-wt. of at least one active substance,
and optionally further auxiliary substances.

4. Administration form according to one or more of the preceding Claims, characterized in that the film former(s) soluble in polar solvents belong(s) to the group of the polyvinyl pyrrolidones, polyvinyl alcohols, polyacrylic acids, polymethacrylic acids, the celluloses, or derivatives thereof.

5. Administration form according to one or more of the preceding Claims, characterized in that the film former(s) is/are soluble in water, ethanol, isopropanol, ethyl acetate or in mixtures thereof.

6. Administration form according to one or more of the preceding Claims, characterized in that the gel former(s) belong(s) to the natural or semi-synthetic polymers of the group of plant polysaccharides.

7. Administration form according to one or more of the preceding Claims, characterized in that the gel former(s) is/are selected from the group of alginates, pectins, carrageenans, tragacanth, xanthene, cellulose derivatives, starch or derivatives thereof, galactoglucomannan or derivatives thereof, chitosan or derivatives thereof.

8. Administration form according to one or more of the preceding Claims, characterized in that the plasticizer(s) is/are selected from the group of the compounds glycerol, sorbitol, mannitol, low-molecular polyethylene glycols, low-molecular polypropylene glycols, citric acid ester, tartaric acid, glyceryl ester, phthalic acid ester, and or tensides.

9. Administration form according to one or more of the preceding Claims, characterized in that the film or sheet contains further auxiliary substances, in particular, penetration enhancers, preserving agents, pH regulators, antioxidants, emulsion stabilising agents, fillers, perfuming agents and/or colourants.

10. Administration form according to one or more of the preceding Claims, characterized in that the film or sheet contains a lipophile internal phase consisting of natural, semi-synthetic or synthetic fats or oils.

11. Administration form according to one or more of the preceding Claims, characterized in that it releases the active substance on the skin upon contact with skin moisture and/or perspiration.

12. Administration form according to one or more of the preceding Claims, characterized in that it is applied to the human skin.

13. Administration form according to one or more of the preceding Claims, characterized in that the film or sheet may be obtained from a solution, dispersion and/or emulsion, by casting, spraying, printing, knife coating or roller application, and subsequent drying.

14. Administration form according to one or more of claims 1 to 12, characterized in that the film or sheet may be obtained from a solution, dispersion and/or emulsion by dosing into films or sheets provided with small cups (e.g. thermoformed blisters) and subsequent drying.
